(19) United States
(12) Patent Application Publication
(10) Pub. No.: US 2008/0177186 A1
(43) Pub. Date: Jul. 24, 2008

(54) Methods and Apparatus for Determining a Treatment Volume of a Fluid Treatment Agent for Treating The Interior of a Blood Vessel

(76) Inventors: Charles R. Slater, Fort Lauderdale, FL (US); Scott L. Jahrmarkt, Miami Beach, FL (US); Brett E. Naglreiter, Austin, TX (US)

Correspondence Address: GORDON & JACOBSON, P.C., 60 LONG RIDGE ROAD, SUITE 407, STAMFORD, CT, 06902, US

(21) Appl. No.: 11/624,431

(22) Filed: Jan. 18, 2007

(51) Int. Cl. A61B 5/02

(52) U.S. Cl. 600/481

(57) ABSTRACT

The methods of the invention include examining a blood vessel to be treated, preferably with the aid of ultrasound, calculating a volume of the portion of the blood vessel, and treating the portion with a volume of treatment agent where the volume of treatment agent is a function of the volume of the portion of the blood vessel. Apparatus for implementing the methods are provided.

### CROSS-REFERENCE TO RELATED APPLICATIONS

This application is related to the following patent applications which are hereby incorporated by reference herein in their entireties:

U.S. Ser. No. 09/898,867 filed Jul. 3, 2001 and entitled “Methods and Apparatus for Sclerosing the Wall of a Varicose Vein”;
U.S. Ser. No. 10/358,523 filed Feb. 5, 2003 and entitled “Methods and Apparatus for Sclerosing the Wall of a Varicose Vein”;
U.S. Ser. No. 10/544,082 filed Jul. 28, 2005 and entitled “Methods and apparatus for Treating the Interior of a Blood Vessel”;
U.S. Ser. No. 10/922,221 filed Aug. 19, 2004 and entitled “Occludable Intravascular Catheter for Drug Delivery and Method of Using the Same”;
U.S. Ser. No. 10/922,123 filed Aug. 19, 2004 and entitled “Methods and Apparatus for Treating the Interior of a Blood Vessel”; and
PCT/US/06/01458 entitled “Valve System for a Medical Device Having an Inflatable Member”.

### BACKGROUND OF THE INVENTION

1. Field of the Invention

This invention relates broadly to the treatment of blood vessels. More particularly, this invention relates to methods and apparatus for determining the correct treatment volume for a fluid treatment agent.

2. State of the Art

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sac or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

Incompetence in the venous system can result from vein dilation. Separation of the cusps of the venous valve at the commissure may occur as a result. Two venous diseases which often involve vein dilation are varicose veins and chronic venous insufficiency.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

The applicants' related patent applications which have been previously incorporated by reference herein disclose methods and apparatus for treating blood vessels, in particular insufficient blood vessels including varicose veins, with injectable treatment fluids. The apparatus generally include a catheter having an occlusion element such as a balloon and an infusion catheter. The occlusion balloon is delivered into the blood vessel through an incision to the place where treatment will commence and is inflated. The infusion catheter then infuses treating fluid into the blood vessel along a specific length of the vessel; typically, the length from the balloon to the incision. Because of different lengths and diameters of blood vessels being treated, catheter treating apparatus of different lengths and diameters may be provided. Also, since blood vessels vary in diameter, the balloon inflation volume should be selected accordingly.

Another parameter involved in the treatment of the vein is the amount of treating fluid to be injected. Prior art techniques tend to use large amounts of sclerosing agent as there has been no mechanism to determine the amount of agent required using the treating methods of the prior art. However, it is generally desirable to avoid using more medication than is necessary to obtain the desired result.

### SUMMARY OF THE INVENTION

It is therefore an object of the invention to provide methods and apparatus for determining a treatment volume of a fluid treatment agent for treating the interior of a blood vessel.

It is another object of the invention to provide methods and apparatus for determining the correct length and diameter of a treating catheter for a particular blood vessel.

It is a further object of the invention to provide methods and apparatus for determining the correct inflation volume for an occlusion balloon.

In accord with these objects, which will be discussed in detail below, the methods of the invention include the steps of measuring a blood vessel to be treated at multiple locations, preferably with the aid of ultrasound, and calculating a treatment volume therefrom. According to one embodiment of the invention, the diameter of the blood vessel to be treated is measured at regular locations (e.g., every 4 cms) along the treatment length, thereby effectively defining “treatment segments”. The volume of the blood vessel is then calculated for each segment using an average of the starting and ending cross sectional areas and the length of the segment. The total of the segment volumes is used to determine the treatment volume.

According to another embodiment of the invention, the blood vessel volume can be calculated by measuring the blood vessel at three locations and determining whether the blood vessel has a generally constant diameter along its length, a decreasing diameter along its length, or an increasing diameter. In particular, the length of the treatment is measured and the diameters of the treatment midpoint and end points are measured. The volume of the blood vessel is calculated using the cross sectional areas at each of the three points and the length. The blood vessel volume is then used to determine the treatment volume

With the blood vessel volume calculation, a determination can be made as to the preferred volume of treating fluid to be infused into the blood vessel. According to a presently preferred embodiment, the volume of treating fluid 1 cc less than 70% of the blood vessel volume. This volume is derived from the expected vasoconstriction of the blood vessel when the treating device (catheter) is inserted, and the priming volume of the treating catheter itself (typically about 1 cc). While the methods have been reduced to practice for the treatment of varicose veins, it is believed that the methods may also be applied to other blood vessel treatments.

At will also be appreciated that with the data collected regarding vessel diameters and treatment length, a determination can be made as to the preferred size of the treating device and the preferred inflation volume of the occlusion balloon.

An apparatus according to the invention includes a data input device, a processor with associated memory for storing data and instructions, and an output device. The apparatus of the invention has been reduced to practice in two embodiments: one is software that runs on a handheld computer (e.g., PALM PILOT) and the other is software that runs on a WINDOWS PC.

Additional objects and advantages of the invention will become apparent to those skilled in the art upon reference to the detailed description taken in conjunction with the provided figures.

### BRIEF DESCRIPTION OF THE DRAWINGS

FIG. 1 is a schematic diagram of a preferred infusion catheter system for treatment of insufficient veins;

FIG. 2 is a high level flow chart illustrating the methods of the invention; and

FIG. 3 is a high level block diagram of an apparatus according to the invention.

### DETAILED DESCRIPTION OF THE PREFERRED EMBODIMENTS

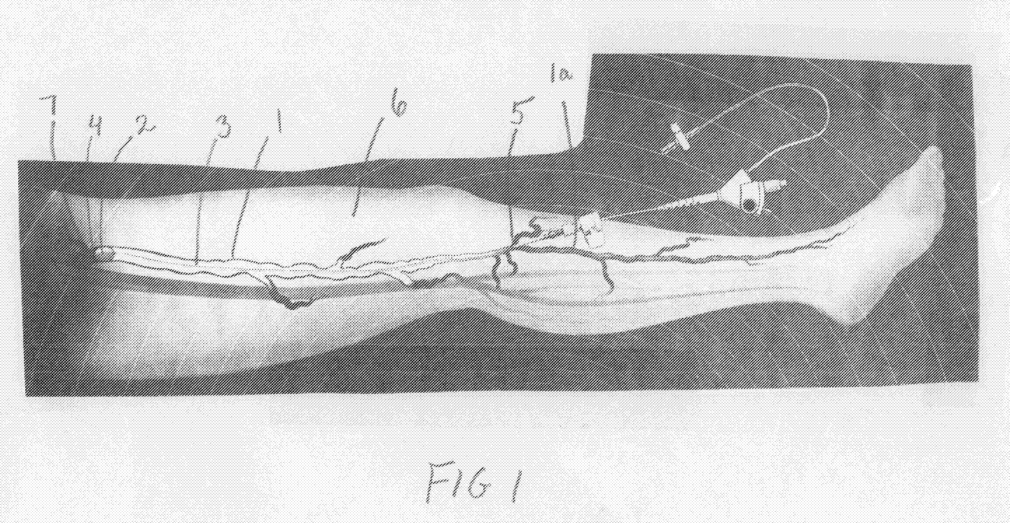

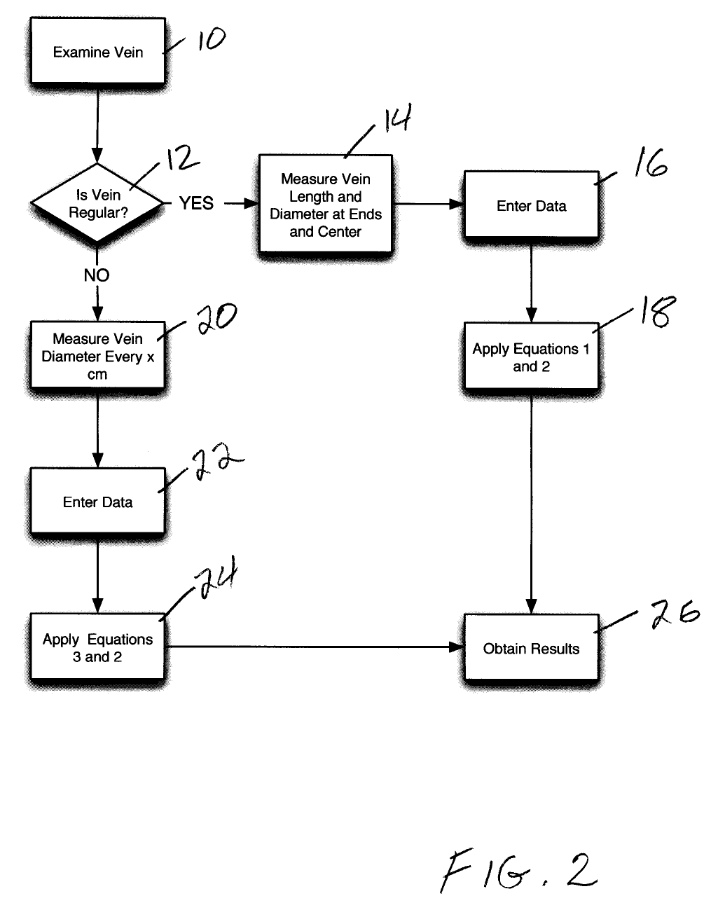

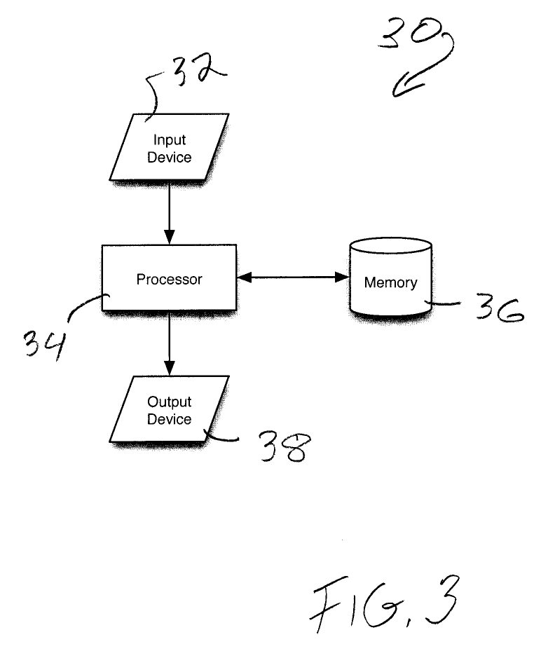

As set forth in the previously incorporated related applications, insufficient (e.g., varicose) veins may be treated using an infusion catheter system. In particular, as seen in FIG. 1, a varicose portion 1 of a greater saphenous vein 1a is treated using an infusion catheter system which includes a distal balloon (occlusion) element 2 and an infusion catheter 3 which includes infusion holes (not shown) and a radiopaque catheter tip 4. According to the treatment method, an incision 5 is made in the leg 6 at the start of the vascular segment to be treated, the infusion catheter 3 is primed with treating agent, and the primed infusion catheter 3 is inserted into the incision and up the vein 1 to a terminal location 7 (in this case just short of the femoral junction). When the tip of the catheter is properly located, the balloon 2 is inflated to occlude the vein portion 1 at location 7, and an appropriate amount of treating agent (as discussed below with reference to FIGS. 2 and 3) is injected into the infusion catheter 3 in order to treat the vein. After treatment, the balloon 2 is deflated, the catheter 3 is removed from the vein 1, and the leg 2 is wrapped.

Turning now to FIG. 2, the methods of the invention are seen for determining the appropriate amount of treating agent to be used in the treatment. Thus, prior to treatment, the blood vessel to be treated is examined at 10, preferably with the aid of ultrasound. The ultrasound may be applied internal or external the vessel. In any event, the ultrasound or other examination technique should be capable of ascertaining the diameter of the blood vessel at various points along the length of the treatment of the vessel. Based on the examination, a determination is made at 12 as to whether the blood vessel has a relatively constant diameter along its length, a generally decreasing diameter, or a generally increasing diameter. If the blood vessel has a generally constant diameter, or a generally decreasing diameter, or a generally increasing diameter, for purposes of the invention it is considered “regular”. Otherwise it is considered “irregular”.

In the case of a regular blood vessel, the length of the treatment is measured or defined, and the diameters of the blood vessel at the treatment midpoint and the end points are located at 14 and entered at 16 into an appropriate apparatus such as described below with reference to FIG. 3. The volume V of the blood vessel is then calculated at 18 using the average cross sectional area at each of the three points and the length according to

$$V = \frac{π {r}_{1}^{2} + π {r}_{2}^{2} + π {r}_{3}^{2}}{3} ( l ) \qquad ( 1 a )$$

where r denotes a radius (one-half the diameter), the subscript indices 1 and 3 denote endpoint locations, and the index 2 denotes a midpoint location, and l is the treatment length. Alternatively, the volume may be calculated at 18 according to:

$$V = ( \frac{π {r}_{1}^{2} + π {r}_{2}^{2}}{2} ) ( l / 2 ) + ( \frac{π {r}_{2}^{2} + π {r}_{3}^{2}}{2} ) ( l / 2 ) \qquad ( 1 b )$$

According to the presently preferred embodiment, the treating fluid (agent) volume AgentV is then calculated according to

AgentV=(0.7V)−1 cc (2)

Equation 2 takes into consideration the priming volume of the treating tool (approximately 1 cc) and the typical vasoconstriction of the blood vessel (approximately 30%) when the treating tool is inserted. According to the invention, a treating fluid volume is substantially equal to the volume AgentV if it is within 10% of the determined value of equation 2.

In the case of an irregular blood vessel, the diameter preferably is measured at regular intervals (e.g., every x cm) at 20, where x is preferably 4. The data pairs (diameter and cm location) are entered at 22. The volume of the blood vessel is calculated at 24 for each segment using the average of the starting and ending cross sectional areas and the segment length, and the segment volumes are then totaled. The calculations are shown generally at 24. The blood vessel volume calculation is performed according to

$$V = {\sum }_{i = 1}^{i = n - 1} \frac{π {r}_{i}^{2} + π {r}_{i + 1}^{2}}{2} ( {l}_{i + 1 , i} ) \qquad ( 3 )$$

where the subscript couplet for the length identifies the segment starting and ending points. The treating fluid volume AgentV is then calculated according to equation (2).

According to another aspect of the invention, with the data (diameters and length) collected, a determination can be made as to the preferred size of the treating device and the preferred inflation volume of the occlusion balloon. For example, the inflation volume of the balloon can be taken from a chart such as Chart 1: Vessel dia (mm) inflation vol (mL)

CHART 1

| | |
|---|---|
| 3.0 | 0.2 |
| 4.0 | 0.3 |
| 5.0 | 0.5 |
| 6.0 | 0.8 |
| 7.0 | 1.1 |
| 8.0 | 1.4 |
| 9.0 | 1.7 |
| 10.0 | 2.2 |
| 11.0 | 2.6 |
| 12.0 | 3.1 |
| 13.0 | 3.6 |
| 14.0 | 4.2 |
| 15.0 | 4.8 |
| 16.0 | 5.5 |
| 17.0 | 6.2 |
| 18.0 | 6.9 |
| 19.0 | 7.7 |

While the methods have been reduced to practice for the treatment of a varicose great saphenous vein, it is believed that the methods may also be applied to other blood vessel treatments.

Turning now to FIG. 3, an apparatus 30 according to the invention includes a data input device 32, a processor 34 with associated memory 36 for storing data and instructions, and an output device 38. The apparatus of the invention has been reduced to practice in two embodiments: one is software that runs on a handheld computer such as a PALM PILOT and the other is software that runs on a WINDOWS PC.

According to alternate embodiment of the apparatus, the memory 36 contains a lookup table which correlates blood vessel length and diameter to stock catheter sizes and the output device displays treatment volume, catheter stock number, and balloon inflation volume.

There have been described and illustrated herein several embodiments of methods and apparatus for determining the volume of a blood vessel and the volume of agent used to treat the blood vessel. While particular embodiments of the invention have been described, it is not intended that the invention be limited thereto, as it is intended that the invention be as broad in scope as the art will allow and that the specification be read likewise. Thus, while a flow chart which incorporates two methods of measuring blood vessel volume has been presented, it will be appreciated that no decisions as to whether a vein is regular or not need be made, and a measurement of the vein at multiple locations can be used instead. Also, the determination of whether a vein is regular or not can be made automatically from the three measurements at the proximal and distal locations and the midpoint; e.g., if the midpoint is the largest measurement or is substantially equal in diameter to only one of the end points, the vein is considered irregular. Of course, other criteria could be utilized to determine regularity. Further, while particular equations were used to find the volume of the vein portion, other equations could be used. Likewise, while a particular equation was used to find a treatment volume as a function of the vein portion volume, other equations could be used. In addition, while the preferred embodiment of the invention suggested measuring a diameter of the vein portion every 4 cm, the measurements could be made at other equal or non-equal intervals.

It will also be appreciated that while particular hardware and software implementations have been disclosed, that other embodiments could perform the methods as well. For example, the methods of the invention could be embodied in a dedicated processor rather than a general purpose processor. Further, the input device could be speech recognition, handwriting recognition, keypad or other. The output could be audio, video, or both. An output display could be LCD, CRT, LEDs or other. It will therefore be appreciated by those skilled in the art that yet other modifications could be made to the provided invention without deviating from its spirit and scope as claimed.

What is claimed is:

1. A method for determining a treatment volume of a fluid treatment agent for treating the interior of a portion of a blood vessel, said method comprising:
obtaining an indication of a volume V of the portion of the blood vessel to be treated; and
determining said treatment volume as a function of said indication.

2. A method according to claim 1, wherein:
said function causes said treatment volume to be less than said volume V.

3. A method according to claim 2, wherein:
said function includes multiplying said volume V by a fraction which provides a fractional volume.

4. A method according to claim 3, wherein:
said function includes subtracting a value from said fractional volume.

5. A method according to claim 1, wherein:
said treatment volume is substantially equal to 0.7V-1 cc.

6. A method according to claim 1, wherein:
said obtaining includes measuring said blood vessel portion at a plurality of locations.

7. A method according to claim 6, wherein:
said obtaining comprises finding volumes of a plurality of segments of said blood vessel portion.

8. A method according to claim 7, wherein:
said obtaining comprises adding together said volumes of a plurality of segments.

9. A method according to claim 8, wherein:
said finding volumes of a plurality of segments comprises finding indications of diameters of said blood vessel portion at said plurality of locations.

10. A method according to claim 9, wherein:
said finding volumes comprises using said indications of diameters and using lengths of said plurality of segments to find said volumes of said plurality of segments.

11. A method according to claim 7, wherein:
said plurality of locations includes at least a first endpoint, a second endpoint, and a midpoint of said portion of said blood vessel.

12. A method according to claim 11, wherein:
said plurality of locations includes additional locations between said first endpoint and said second endpoint.

13. A method according to claim 11, further comprising:
examining said portion of said blood vessel to determine whether it is a regular blood vessel in that it has a generally constant, generally increasing, or generally decreasing diameter.

14. A method according to claim 13, wherein:
if said portion of said blood vessel is regular, said plurality of locations includes only said first endpoint, said second endpoint, and said midpoint of said portion of said blood vessel.

15. A method for treating a portion of a blood vessel with a sclerosing agent, comprising:
determining indications of diameters of the blood vessel at a plurality of locations along said blood vessel;
using said indications, determining a sclerosing agent treatment volume; and
using said sclerosing agent treatment volume to treat said portion of said blood vessel.

16. A method according to claim 15, wherein:
said using said indications comprises finding a blood vessel volume V for said portion of said blood vessel.

17. A method according to claim 16, wherein:
said finding a blood vessel volume comprises
finding blood vessel segment volumes for segments defined by pairs of adjacent locations by determining cross sectional areas of the portion of the blood vessel at said plurality of locations, averaging the adjacent cross sectional areas and multiplying the average by the distance between them, and
totaling the volumes.

18. A method according to claim 16, wherein:
said determining a sclerosing agent treatment volume comprises determining said sclerosing agent treatment volume as a function of said blood vessel volume V.

19. A method according to 18, wherein:
said function is a fraction of said blood vessel volume V minus a volume.

20. A method according to claim 15, wherein:
said plurality of locations includes first and second endpoints of said portion of said blood vessel, and at least one location therebetween.

21. A method according to claim 20, wherein:
said at least one location therebetween comprises a plurality of locations therebetween.

22. A method according to claim 15, further comprising:
prior to said using, occluding said portion of said blood vessel at an endpoint thereof.

23. An apparatus for calculating the treatment volume of a treatment agent to be dispensed inside a blood vessel for treating a portion of the blood vessel, comprising:
input means for receiving data related to the length of said portion of the blood vessel and diameters at least three locations along the length of said portion of the blood vessel;
processor means for calculating the volume of the blood vessel based on said data and for calculating the treatment volume based on the volume of the blood vessel; and
output means for conveying the treatment volume, said output means being coupled to the processor means.

24. An apparatus according to claim 23, wherein:
said processor means calculates said treatment volume as a function of said volume of the blood vessel.

25. An apparatus according to claim 24, wherein:
said function is a fraction of said blood vessel volume minus a predetermined volume.

26. An apparatus according to claim 24, wherein:
said plurality of locations includes first and second endpoints of said portion of said blood vessel, and at least one location therebetween.
